SEQUENCE LISTING (29 sequences)

SEQ ID NO: 1 - DNA, 3162 bp, Staphylococcus aureus
  CDS (1)..(3162)
atgaagaggaactacatcctcggactcgatattggaatcacctctgtgggatacggaatcatcgattacgagactagggatgtgatcgatgctggtgtgagactcttcaaagaggctaacgttgagaacaacgagggaagaaggtctaagaggggagctagaaggctcaagagaagaagaaggcacagaatccagagggtgaagaagctcctcttcgattacaacctcctcaccgatcactctgagctttctggaatcaacccttacgaggcaagagtgaagggactctctcagaagttgtctgaggaagagttctctgctgctttgctccaccttgctaagagaaggggtgtgcataacgtgaacgaggtggaagaggataccggaaacgagctttctaccaaagagcagatcagtaggaactctaaggctctcgaagagaagtacgtggcagagcttcagcttgagagactcaagaaagatggtgaggtgaggggatctatcaacaggttcaagacctctgattacgtgaaagaagctaagcagctcctcaaggtgcagaaggcttaccatcagctcgatcagtctttcatcgatacctacatcgatctcttggagactagaaggacctactacgagggacctggtgaaggatctccattcggatggaaggatattaaggaatggtacgagatgctcatgggacactgcacttacttccctgaggaactcagatcagtgaagtacgcttacaacgctgatctctacaacgctcttaacgatctcaacaacctcgtgatcaccagggatgagaacgagaagttggagtactatgagaagttccagatcatcgagaacgtgttcaagcaaaagaagaagcctaccctcaagcagatcgctaaagagatccttgttaacgaagaagatattaagggatacagggtgacctctaccggaaagcctgagttcactaacctcaaggtttaccacgatattaaggatattaccgctagaaaagagattattgagaacgctgagcttctcgatcaaatcgctaagatccttaccatctaccagtcatctgaggatattcaagaagagttgaccaacctcaactcagagcttacccaagaggaaatcgagcaaatctctaaccttaagggttacaccggaacccacaacctctcactcaaggctatcaacctcatcctcgatgagctttggcacaccaacgataaccagatcgcaatcttcaacagactcaagctcgtgcctaagaaagtggatctctctcagcagaaagagattcctaccaccctcgtggatgatttcatcctctcacctgtggtgaagagatcattcatccagtctatcaaggtgatcaacgctattatcaagaaatacggactccctaacgatattatcatcgagttggctagggaaaagaactcaaaggatgctcaaaagatgatcaacgagatgcaaaagaggaacaggcagaccaacgagaggatcgaggaaatcatcaggaccaccggaaaagagaacgctaagtaccttatcgagaagatcaagctccacgatatgcaagagggaaagtgcctctactctcttgaggctatccctcttgaggatctccttaacaaccctttcaactacgaagtggatcatatcatccctagatcagtgtctttcgataactctttcaacaacaaggtgctcgtgaagcaagaagaaaactctaagaagggaaacaggacccctttccagtacctctcttcatctgattctaagatcagttacgagacattcaagaaacacatcctcaacctcgctaagggaaagggtaggatctcaaagacaaagaaagagtacctccttgaggaaagggatattaacaggttctctgttcagaaggatttcatcaacagaaaccttgtggataccaggtacgctaccaggggtcttatgaaccttctcaggtcttacttcagggtgaacaaccttgatgtgaaggtgaagtcaatcaacggtggattcacctcattccttagaagaaaatggaagtttaagaaagagaggaacaagggttataagcaccacgctgaggatgctctcattatcgctaacgcagatttcattttcaaagagtggaagaaacttgataaggctaaaaaggtgatggaaaaccagatgttcgaggaaaagcaggctgagtctatgcctgagatcgagactgagcaagagtacaaagagattttcatcacccctcaccagatcaagcacatcaaagatttcaaggattataagtactctcacagagtggataagaagccaaacagagagcttatcaacgataccctctactcaaccagaaaggatgataagggtaacaccctcatcgtgaacaatctcaacggactctacgataaggataacgataagctcaagaagctcatcaacaagtctccagagaagttgctcatgtaccaccacgatcctcagacctaccaaaagctcaagttgatcatggaacagtacggtgatgagaagaaccctctctacaagtactacgaagagactggaaactacctcaccaagtactcaaaaaaggataatggacctgtgatcaagaagattaagtattacggaaacaagctcaacgcacacctcgatattactgatgattaccctaactctaggaacaaagtggtgaagctctcacttaagccttacaggttcgatgtgtacctcgataacggtgtgtacaagttcgtgaccgtgaagaacctcgatgttattaagaaagaaaactattacgaggtgaacagtaagtgctacgaggaagctaagaagttgaagaagatcagtaaccaggcagagttcattgcttcattctacaacaacgatcttatcaagattaacggtgagctttacagggttatcggagtgaacaacgatttgctcaacaggatcgaagtgaacatgattgatattacctacagggaataccttgagaatatgaacgataagaggcctcctaggatcatcaagactatcgcttctaagacccagagtatcaagaagtactctaccgatattctcggaaacctctacgaggttaagtcaaagaagcaccctcagatcattaagaagggttga

SEQ ID NO: 2 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - DNA, 37 bp, Artificial Sequence
  Synthetic sequence, ribozyme sequence (HamRz)
ctgatgaggccgaaaggccgaaactccgtaaggagtc

SEQ ID NO: 4 - DNA, 1668 bp, Artificial Sequence
  Synthetic sequence, a Luc gene in which Cas9 target sequence is inserted
  misc_structure (6)..(28): inserted sequence
  misc_structure (6)..(25): Cas9 target sequence
atgagcgcttgtttcggcgtgggtatgggagagagagaagaacgtggtgtacggccccgagcccaagcaccctctgggcaacttcaccgccggcgagatgctgtacaacgctctgcacaagcactcccacatcccccaggccatcctggacgtgatgggcaacgagtccctttcctaccaggagttcttcgacactactgtgaagctgggccagagcctccagaactgtggctacaagatgaacgatgtcgtgtcgatctgtgcagagaacaacaagagattcttcatccccatcatctccgcctggtacatcggcatggtggtggcccctgtgaacgaggactatatcccagacgagctgtgtaaagtgaccggcatctccaagccgatcctggtcttcaccactaggaagatcctgcctaaggttttggaggttaaagacagaaccaactacataaagaggatcatcatactggactctgaagagaacctgctgggctgcgagagcctgcacaacttcatgtccaggtactccgacaacaacctccaaacattcaagcctctgcactacgaccctgtggaccaggtagccgccatcctgtgctcctccggcacaaccggcctgcctaaaggcgtgatgcagacccacaggaacatctgtgtgagactcacacacgcatctgaccccagagtgggtacacaactcatccccggcgtatccgtgctggcctacctgccattcttccacgccttcggcttcagtatcaacctgggctatttcatggtgggcctgagagtggtgatgctccgaaggtttaaccaggaggtgttcctgaaggccatccaggactacgaggtgaggagcgtgatcaacgttccctccacaatcctgttcctgtccaagagccctctggtggacaagtacgacctatccaccctggcggagctgtgctgtggagccgctcctctggcgaaggaggtggccgagatcgccgtgaagaggctgaacctgccagggatacggtgtggctacggtctaacagagtctacctccgccaacatccatactctgcacaacgagttcaagtccggctccctgggcaaggtgacaccttacatggccgccaagatcatcgacaggaacaccggcgaggccctgggtccaaaccaggtgggcgagctgtgcatctggggacctatggtaacaaaaggctatgtgaacaacccacaggctactaaggaggccatcgacgacgacggctggctgcactctggcgacttcggctactacgacgaggacgagtatttctacatcgtggaccggtacaaggagctgatcaaatacaagggctatcaggtcgcccctgtggagctggaggagatcctccttcagcacccaggcatcagggacgtggccgtcgtgggtatccctgacatcgaggccggcgagctgccagccggcttcgtggtgaagcagcccggcgcccaactcaccgctaaggaggtgtacgacttcctggcccagagggtgtctcactccaagtacctgaggggcggcgtaaggttcgtggactctatccccaggaacgtgacaggcaagattagtcgaaaagagctgagggaggccctgatggagaaggcttctaagctgtaatctagagcggccgc

SEQ ID NO: 5 - DNA, 4107 bp, Streptococcus pyogenes
  CDS (1)..(4104)
atggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattga

SEQ ID NO: 6 - protein, 1368 aa, Streptococcus pyogenes [sequence omitted]

SEQ ID NO: 7 - DNA, 88 bp, Artificial Sequence
  Synthetic sequence, AgeToMVRz1
acggaccggtgaaacaagcgctgatgagtccgtgaggacgaaactataggaaaggaattcctatagtccgcttgtttcggcgtgggta

SEQ ID NO: 8 - DNA, 78 bp, Artificial Sequence
  Synthetic sequence, AgeToMVRz2
acggaccggtaacaagcgctgatgaggccgaaaggccgaaacccggtatcccgggttccgcttgtttcggcgtgggta

SEQ ID NO: 9 - DNA, 71 bp, Artificial Sequence
  Synthetic sequence, AgeToMVRz3
acggaccggtagcgctgatgaggccgaaaggccgaaactccgtaaggagtccgcttgtttcggcgtgggta

SEQ ID NO: 10 - DNA, 73 bp, Artificial Sequence
  Synthetic sequence, AgeToMVRz4
acggaccggtcaagcgctgatgaggccgaaaggccgaaactccgtaaggagtccgcttgtttcggcgtgggta

SEQ ID NO: 11 - DNA, 79 bp, Artificial Sequence
  Synthetic sequence, AgeToMVRz5
acggaccggtgaaacaagcgctgatgagtccgtgaggacgaaacccggagtcccgggtccgcttgtttcggcgtgggta

SEQ ID NO: 12 - DNA, 30 bp, Artificial Sequence
  Synthetic sequence, NoRz
acggagctcttgcgatgcctaacaagccag

SEQ ID NO: 13 - DNA, 70 bp, Artificial Sequence
  Synthetic sequence, Rz3
acggagcgacaactgatgaggccgaaaggccgaaactccgtaaggagtcttgcgatgcctaacaagccag

SEQ ID NO: 14 - DNA, 71 bp, Artificial Sequence
  Synthetic sequence, Rz4
acggagcgagcaactgatgaggccgaaaggccgaaactccgtaaggagtcttgcgatgcctaacaagccag

SEQ ID NO: 15 - DNA, 71 bp, Artificial Sequence
  Synthetic sequence, Rz4a
acggagcgagcgactgatgaggccgaaaggccgaaactccgtaaggagtcttgcgatgcctaacaagccag

SEQ ID NO: 16 - DNA, 71 bp, Artificial Sequence
  Synthetic sequence, Rz4b
acggagcgagtaactgatgaggccgaaaggccgaaactccgtaaggagtcttgcgatgcctaacaagccag

SEQ ID NO: 17 - DNA, 72 bp, Artificial Sequence
  Synthetic sequence, Rz5a
acggagcgacgcgactgatgaggccgaaaggccgaaactccgtaaggagtcttgcgatgcctaacaagccag

SEQ ID NO: 18 - DNA, 72 bp, Artificial Sequence
  Synthetic sequence, Rz5b
acggagcgacgtaactgatgaggccgaaaggccgaaactccgtaaggagtcttgcgatgcctaacaagccag

SEQ ID NO: 19 - DNA, 21 bp, Artificial Sequence
  Synthetic sequence, encoding a guide RNA
taaggatcgaattgagggtac

SEQ ID NO: 20 - DNA, 46 bp, Artificial Sequence
  Synthetic sequence, 5′Rz
aaatctttactgatgaggccgaaaggccgaaactccgtaaggagtc

SEQ ID NO: 21 - DNA, 58 bp, Artificial Sequence
  Synthetic sequence, 3′Rz1
ggcactcgtgaagagcaacctcctcgtggtgagtgttgggtaacgctaatagcttttt

SEQ ID NO: 22 - DNA, 57 bp, Artificial Sequence
  Synthetic sequence, 3′Rz2
ggcactcgtgaagagcaacctcccgtggtgagtgttgggtaacgctaatagcttttt

SEQ ID NO: 23 - DNA, 58 bp, Artificial Sequence
  Synthetic sequence, 3′Rz3
ggcactcgtgaagagcaacctcctcgtggtgagtgttgggtaacgctaatagttcttt

SEQ ID NO: 24 - DNA, 58 bp, Artificial Sequence
  Synthetic sequence, 3′Rz4
ggcactcgtgaagagcaacctcctcgtggtgggtgttgggtaacgctaatagctcttt

SEQ ID NO: 25 - DNA, 58 bp, Artificial Sequence
  Synthetic sequence, 3′Rz5
ggcactcgtgaagagcaacctcctcgtggtgagtgttgggtaacgctaatagtttttt

SEQ ID NO: 26 - DNA, 58 bp, Artificial Sequence
  Synthetic sequence, 3′Rz6
ggcactcgtgaagagcaacctcctcgtggtgggtgttgggtaacgctaatagttcttt

SEQ ID NO: 27 - DNA, 58 bp, Artificial Sequence
  Synthetic sequence, 3′Rz7
ggcactcgtgaagagcaacctcctcgtggtgggtgttgggtaacgctaatagcttttt

SEQ ID NO: 28 - DNA, 58 bp, Artificial Sequence
  Synthetic sequence, 3′Rz8
ggcactcgtgaagagcaacctcctcgtggtgggtgttgggtaacgctaatagtttttt

SEQ ID NO: 29 - DNA, 58 bp, Artificial Sequence
  Synthetic sequence, 3′Rz9
ggcactcgtgaagagcaacctcctcgtggtgagtgttgggtaacgctaatagctcttt

(19) United States
(12) Patent Application Publication
(10) Pub. No.: US 2019/0359993 A1
(43) Pub. Date: Nov. 28, 2019

(54) METHOD FOR PRODUCING GENOME-EDITED PLANT UTILIZING PLANT VIRUS VECTORS

(71) Applicant: National Agriculture and Food Research Organization, Tsukuba-shi, Ibaraki (JP)

(72) Inventors: Kazuhiro ISHIBASHI, Tsukuba-shi (JP); Hirotaka ARIGA, Tsukuba-shi (JP); Seiichi TOKI, Tsukuba-shi (JP); Hidetaka KAYA, Tsukuba-shi (JP)

(73) Assignee: National Agriculture and Food Research Organization, Tsukuba-shi, Ibaraki (JP)

(21) Appl. No.: 16/486,082

(22) PCT Filed: Feb. 14, 2018
(86) PCT No.: PCT/JP2018/005085

§ 371 Date: Aug. 14, 2019

(30) Foreign Application Priority Data
Feb. 15, 2017 (JP) 2017-025976

(51) Int. Cl. C12N 15/82; C12N 5/14

(52) U.S. Cl. CPC C12N 15/8213; C12N 5/14

(57) ABSTRACT

A combination of virus vectors for genome editing is formed by arranging a polynucleotide encoding a split genome editing enzyme in each of a Tobamovirus vector and a Potexvirus vector and arranging a polynucleotide encoding a guide RNA in one of the vectors. It is found that when these virus vectors are introduced into a plant cell, a complex of a functional Cas9 protein and the guide RNA is formed in the plant cell, and a genome is edited in a target site-specific manner.

### TECHNICAL FIELD

The present invention relates to a method for producing a genome-edited plant cell and plant utilizing a plurality of types of plant positive-sense single-stranded RNA virus vectors, and a kit for use in the method.

### BACKGROUND ART

The genome editing technology is a technology of creating new cells and cultivars by introducing mutations to target sites of particular genes to modify the activities of the encoding proteins (for example, substitution from an active type to an inactive type or substitution from an inactive type to an active type). This technology makes it possible to create cultivars and lines that do not hold exogenous genes since mutations are simply introduced into endogenous genes, and in this point is different from the conventional genetic recombination technology (NPL 1).

In the genome editing technology, in order to provide two features, a site specificity on the genome and genome alteration, a nuclease (nucleic acid (DNA) cleaving enzyme) provided with a site specificity is generally utilized. As such nucleases, since 2005, following the first generation ZFNs (Zinc Finger Nucleases), the second and third generation genome editing technologies, such as TALENs (Transcription Activator Like Effector Nucleases) and CRISPR-Cas9 (Clustered Regularly Interspaced Short Palindromic Repeats CRISPR-Associated Proteins 9), have been developed one after another (NPL 2). To provide a site specificity to a nuclease, ZFNs and TALENs utilize sequence recognition domains (ZF domain and TALE domain) that bind to a target DNA while CRISPR/Cas9 utilizes an RNA (guide RNA) that has a sequence complementary with a target DNA.

When an artificial nuclease for genome editing, such as TALENs, is utilized to breed a crop, methods for introducing artificial nuclease genes through genetic recombination technologies such as the agrobacterium method have been mainstream in the case of plants (NPL 3). However, since in the agrobacterium method, an artificial nuclease gene is integrated in the genome DNA of the target plant, it is necessary to remove the artificial nuclease gene, which becomes unnecessary after the target gene of the plant is edited. In this case, if the plant allows crossing, the gene of the protein for genome editing can be removed; however, there are also many crops that virtually do not allow removal of unnecessary genes through crossing, such as vegetative-propagation plants and arboreous plants.

From this background, the developments of technologies have been conducted that allow genome editing by directly introducing artificial nucleases as proteins into cells without integrating genes into genomes. However, since these technologies require protoplast transformation, and because of some other reasons, they are applicable only to limited plant cultivars (NPLs 4 and 5). Likewise, there was a report that a target gene was successfully mutated by directly introducing an RNP (CRISPR/Cas9 protein-RNA complex) into a corn embryo by means of a particle gun method (NPL 6). However, since the particle gun method is only capable of regenerating individuals of limited plants even in the case of normal gene recombination, it can be considered to be more difficult to acquire regenerated individuals in the case of genome editing.

In addition, there has been an attempt to conduct genome editing of plants via viruses (NPL 7). However, while there is a limitation in size of genes capable of being expressed from virus vectors, the size of enzymes for genome editing are large, which has made it difficult to conduct genome editing of plants.

### CITATION LIST

### Non Patent Literature

[NPL 1] Voytas, Annu. Rev. Plant Biol. 64:327-350 (2013)

[NPL 2] Doyle et al., Trends Cell Biol 23:390-398 (2013)

[NPL 3] Endo et al., Methods in Molecular Biology Volume 1469 pp. 123-135 (2016)

[NPL 4] Woo et al., Nature Biotech. 33:1162-1164 (2016)

[NPL 5] Subburaj et al., Plant Cell Rep. 35:1535-1544 (2016)

[NPL 6] Svitashev et al., Nature Communication 7:13274 (2016)

[NPL 7] Ali et al., Molecular Plant 8:1288-1291 (2015)

### SUMMARY OF INVENTION

### Technical Problem

The present invention has been made in view of the problems of the above-described conventional technologies, and has an object to provide a method capable of conducting genome editing of a plant using plant virus vectors without integrating a genome editing enzyme gene into a genome.

### Solution to Problem

Considering the fact that the size of a gene that can be expressed from a plant virus vector is limited, the present inventors first developed an idea of splitting a genome editing enzyme, mounting the split fragments of the genome editing enzyme on a plurality of plant virus vectors, and after expression in the plant cell, forming a functional genome editing enzyme through assembling. Considering the possibility that if plant virus vectors of the same genus are utilized for expression of the respective fragments, this could act exclusively in the plant cell, the present inventors decided to employ different plant virus vectors. In addition, to prevent the mounted gene from being integrated into the plant genome, the present inventors decided to employ plant positive-sense single-stranded RNA virus vectors as the plant virus vectors.

Based on the above-described idea, the present inventors prepared a combination of virus vectors for genome editing by using a Tobamovirus vector and a Potexvirus vector as an example of a combination of plant positive-sense single-stranded RNA virus vectors, and arranging a polynucleotide encoding a split genome editing enzyme in each of the virus vectors and arranging a polynucleotide encoding a guide RNA in at least one of the virus vectors. Then, when the present inventors introduced the combination of virus vectors into a plant cell, a complex of a functional Cas9 protein and the guide RNA was formed in the plant cell. The present inventors thus found that a genome was edited in a target site-specific manner. Moreover, when the present inventors arranged a self-cleaving ribozyme in the 5′ terminal of the guide RNA and allowed the ribozyme to appropriately cleave the 5′ side of the guide RNA in the plant cell, the present inventors found that the efficiency of editing a genome was significantly enhanced. These findings have led to the completion of the present invention.

The present invention relates to a method for producing a plant cell and a plant in which a genome is edited, utilizing a plurality of types of plant positive-sense single-stranded RNA virus vectors, as well as a kit for use in the method, and more particularly provides the following.

(1) A method for producing a plant cell in which a genome is edited in a site-specific manner, comprising:

introducing a combination of a plurality of types of plant positive-sense single-stranded RNA virus vectors having characteristics (a) and (b) into a plant cell, where
(a) each of the virus vectors contains a polynucleotide encoding a split genome editing enzyme, and
(b) at least one of the virus vectors contains a polynucleotide encoding a guide RNA; and

allowing a complex containing an assembly of the split genome editing enzymes and the guide RNA to be formed in the plant cell and allowing the complex to edit the genome in a site-specific manner.

(2) A method for producing a plant in which a genome is edited in a site-specific manner, comprising:

introducing a combination of a plurality of types of plant positive-sense single-stranded RNA virus vectors having characteristics (a) and (b) into a plant cell, where
(a) each of the virus vectors contains a polynucleotide encoding a split genome editing enzyme, and
(b) at least one of the virus vectors contains a polynucleotide encoding a guide RNA; and

allowing a complex containing an assembly of the split genome editing enzymes and a guide RNA to be formed in a plant cell, allowing the complex to edit the genome in a site-specific manner, and regenerating a plant from the plant cell.

(3) The method according to (1) or (2), wherein

the combination of plant positive-sense single-stranded RNA virus vectors includes a combination of a Tobamovirus vector and a Potexvirus vector.

(4) The method according to any one of (1) to (3), wherein

the genome editing enzyme is a Cas9 protein or a Cpf1 protein.

(5) The method according to any one of (1) to (4), wherein

a polynucleotide encoding a self-cleaving ribozyme is bound to a 5′ terminal of the polynucleotide encoding a guide RNA.

(6) The method according to any one of (1) to (5), wherein

a polynucleotide encoding a self-cleaving ribozyme is bound to a 3′ terminal of the polynucleotide encoding a guide RNA.

(7) A kit for use in the method according to any one of (1) to (6), comprising:

a combination of a plurality of types of plant positive-sense single-stranded RNA virus vectors having characteristics (a) and (b), where

(a) each of the virus vectors contains a polynucleotide encoding a split genome editing enzyme, and

(b) at least one of the virus vectors contains a polynucleotide encoding a guide RNA or a portion for inserting the polynucleotide.

(8) The kit according to (7), wherein

the combination of plant positive-sense single-stranded RNA virus vectors includes a combination of a Tobamovirus vector and a Potexvirus vector.

(9) The kit according to (7) or (8), wherein

the genome editing enzyme is a Cas9 protein or a Cpf1 protein.

(10) The kit according to any one of (7) to (9), wherein

a polynucleotide encoding a self-cleaving ribozyme is bound to a 5′ terminal of the polynucleotide encoding a guide RNA.

(11) The kit according to any one of (7) to (10), wherein

a polynucleotide encoding a self-cleaving ribozyme is bound to a 3′ terminal of the polynucleotide encoding a guide RNA.

### Advantageous Effects of Invention

In the present invention, the split genome editing enzymes and the guide RNA are mounted and used in the combination of a plurality of types of plant positive-sense single-stranded RNA virus vectors, which has made it possible to conduct genome editing of a plant without integrating a genome editing enzyme gene into a plant genome. In addition, arranging the self-cleaving ribozyme in the 5′ side of the guide RNA has made it possible to significantly enhance the genome editing efficiency. The present invention is the world's first example that has succeeded in genome editing of plants using only autonomously replicating virus vectors.

Conventionally, in conducting genome editing of a plant, if the agrobacterium method is utilized for introducing a genome editing enzyme gene into the plant, the gene is integrated into the plant genome. For this reason, it has been necessary to remove an artificial nuclease gene which becomes unnecessary after the genome editing. In this case, if the plant allows crossing, the gene of a genome editing protein can be removed; however, there have been many crops that virtually do not allow removal of unnecessary genes through crossing, such as vegetative-propagation plants and arboreous plants. The present invention makes it possible to conduct genome editing even on such plants without integrating an exogenous gene into the plant genome.

### BRIEF DESCRIPTION OF DRAWINGS

FIG. 1 is diagrams showing expression of CRISPR-gRNAs by ToMV vectors in which (a) shows structures of the ToMV vectors, (b) shows a photograph of electrophoresis of in vitro transcription products, and (c) is a photograph showing results of detecting LUC activities when ToMV vectors having various ribozymes and the gRNA were inoculated into leaves of Nicotiana benthamiana in which SpCas9 and a target sequence (LUC is expressed by cleavage and repair) were transiently introduced. Note that base sequences in FIG. 1 are shown in SEQ ID NOs: 7 to 11 of Sequence Listing in the order from above.

FIG. 2 is diagrams showing expression of CRISPR-gRNAs and genome editing by ToMV vectors, in which (a) shows structures of the ToMV vectors, (b) shows a photograph of electrophoresis of in vitro transcription products, and (c) is a photograph of electrophoresis showing results of inoculating ToMV vectors having various ribozymes and a gRNA for an endogenous PDS gene into leaves of Nicotiana benthamiana in which SaCas9 was transiently introduced, and analyzing the presence or absence of genome editing in the target portions of the genome DNAs by a CAPS method. Note that base sequences in FIG. 2 are shown in SEQ ID NOs: 12 to 18 of Sequence Listing in the order from above.

FIG. 3 is diagrams showing genome editing through coinfection of a ToMV vector and a PVX vector, in which the upper part shows the structures of the ToMV vector and the PVX vector in each of which a split SaCas9 protein was mounted. In the ToMV vector, a gRNA for a PDS gene was further mounted with a ribozyme therebetween. The lower part is a photograph of electrophoresis showing results of inoculating transcription products of the above-described two types of vectors into leaves of Nicotiana benthamiana and analyzing the presence or absence of genome editing in the target portions of the genome DNAs by the CAPS method.

FIG. 4 is a photograph of a redifferentiated shoot of tobacco in which a PDS gene was destroyed by coinfection of the ToMV vector and the PVX vector.

FIG. 5 is diagrams showing expression of CRISPR-gRNAs and genome editing by ToMV vectors, in which (a) shows structures of a gRNA and ribozymes (ribozymes arranged in the 5′ side of the gRNA and ribozymes arranged in the 3′ side of the gRNA) inserted into the ToMV vectors, (b) shows a photograph of electrophoresis showing results of inoculating ToMV vectors having various ribozymes and the gRNA for an endogenous TOM1 gene into leaves of Nicotiana benthamiana in which SaCas9 was transiently introduced, and analyzing the presence or absence of genome editing in the target portions of the genome DNAs by the CAPS method. Note that base sequences in (a) of FIG. 5 are shown in SEQ ID NOs: 19 to 29 of Sequence Listing in the order from above.

FIG. 6 is a photograph of electrophoresis showing results of inoculating ToMV vectors and PVX vectors expressing a split SpCas9 into leaves of Nicotiana benthamiana and analyzing the presence or absence of genome editing in the target portions of the genome DNAs by the CAPS method.

### DESCRIPTION OF EMBODIMENTS

The present invention provides a method for producing a plant cell in which a genome is edited in a site-specific manner.

In the method of the present invention, a plurality of types of plant positive-sense single-stranded RNA virus vectors are introduced into a plant cell.

Herein, the “plant positive-sense single-stranded RNA virus vector” means a vector derived from a plant virus that is a virus having a positive-sense single-stranded RNA as a genome. The positive-sense RNA is different from a negative-sense RNA in that the positive-sense RNA itself functions as an mRNA. The “plant positive-sense single-stranded RNA virus vector” in the present invention is a polynucleotide that has a gene derived from a virus genome and an exogenous gene in an expressible form, and may be either in the form of an RNA (for example, a transcription product of an expression construct in which an exogenous gene is inserted in a cDNA for a viral genome RNA) or in the form of a DNA (for example, an expression construct in which an exogenous gene is inserted in a cDNA for a viral genome RNA). The “combination of plant positive-sense single-stranded RNA virus vectors” used in the present invention is preferably such that the host ranges of the virus vectors overlap each other, have a low pathogenicity, and are capable of stably expressing split genome editing enzymes. In addition, to avoid interference with each other, the combination is preferably a combination of virus vectors derived from plant viruses belonging to different genera. The virus vectors derived from plant viruses belonging to different genera include, for example, a plurality of types of virus vectors selected from the group consisting of Tobamovirus vectors, Potexvirus vectors, Potyvirus vectors, Tobravirus vectors, Tombusvirus vectors, Cucumovirus vectors, Bromovirus vectors, Carmovirus vectors, and Alfamovirus vectors. Herein, the “plurality of types” means two types or more (for example, two types, three types, four types, or the like). The combination is preferably a combination of two types that are a Tobamovirus vector and a Potexvirus vector.

The Tobamovirus vectors include, for example, a tomato mosaic virus (ToMV) vector, a tobacco mosaic virus (TMV) vector, a tobacco mild green mosaic virus (TMGMV) vector, a pepper mild mottle virus (PMMoV) vector, a paprika mild mottle virus (PaMMV) vector, a cucumber green mottle mosaic virus (CGMMV) vector, a kyuri green mottle mosaic virus (KGMMV) vector, a hibiscus latent fort pierce virus (HLFPV) vector, an odontoglossum ringspot virus (ORSV) vector, a rehmannia mosaic virus (ReMV) vector, a Sammon's opuntia virus (SOV) vector, a wasabi mottle virus (WMoV) vector, a youcai mosaic virus (YoMV) vector, a sunn-hemp mosaic virus (SHMV) vector, and the like. The Potexvirus vectors include, for example, a potato virus X (PVX) vector, apotatoaucubamosaicvirus (PAMV) vector, an Alstroemeria virus X (AlsVX) vector, a cactus virus X (CVX) vector, a Cymbidium mosaic virus (CymMV) vector, a hosta virus X (HVX) vector, a lily virus X (LVX) vector, a Narcissus mosaic virus (NMV) vector, a Nerine virus X (NVX) vector, a Plantago asiatica mosaic virus (PlAMV) vector, a strawberry mild yellow edge virus (SMYEV) vector, a tulip virus X (TVX) vector, a white clover mosaic virus (WClMV) vector, a bamboo mosaic virus (BaMV) vector, and the like. The Potyvirus vectors include, for example, a potato virus Y (PVY) vector, a bean common mosaic virus (BCMV) vector, a clover yellow vein virus (ClYVV) vector, an East Asian Passiflora virus (EAPV) vector, a Freesia mosaic virus (FreMV) vector, a Japanese yam mosaic virus (JYMV) vector, a lettuce mosaic virus (LMV) vector, a Maize dwarf mosaic virus (MDMV) vector, an onion yellow dwarf virus (OYDV) vector, a papaya ringspot virus (PRSV) vector, a pepper mottle virus (PepMoV) vector, a Perilla mottle virus (PerMoV) vector, a plum pox virus (PPV) vector, a potato virus A (PVA) vector, a sorghum mosaic virus (SrMV) vector, a soybean mosaic virus (SMV) vector, a sugarcane mosaic virus (SCMV) vector, a tulip mosaic virus (TulMV) vector, a turnip mosaic virus (TuMV) vector, a watermelon mosaic virus (WMV) vector, a zucchini yellow mosaic virus (ZYMV) vector, a tobacco etch virus (TEV) vector, and the like. The Tobravirus vectors include, for example, a tobacco rattle virus (TRV) vector and the like. The Tombusvirus vectors include, for example, a tomato bushy stunt virus (TBSV) vector, an eggplant mottled crinkle virus (EMCV) vector, a grapevine Algerian latent virus (GALV) vector, and the like. The Cucumovirus vectors include, for example, a cucumber mosaic virus (CMV) vector, a peanut stunt virus (PSV) vector, a tomato aspermy virus (TAV) vector, and the like. The Bromovirus vectors include, for example, a brome mosaic virus (BMV) vector, a cowpea chlorotic mottle virus (CCMV) vector, and the like. The Carmovirus vectors include, for example, a carnation mottle virus (CarMV) vector, a melon necrotic spot virus (MNSV) vector, a pea stem necrotic virus (PSNV) vector, a turnip crinkle virus (TCV) vector, and the like. The Alfamovirus vectors include, for example, an alfalfa mosaic virus (AMV) vector, and the like.

The “combination of a plurality of types of plant positive-sense single-stranded RNA virus vectors” in the present invention has characteristics (a) and (b) described below.

(a) each of the virus vectors contains a polynucleotide encoding a split genome editing enzyme

(b) at least one of the virus vectors contains a polynucleotide encoding a guide RNA

The “genome editing enzyme” in the present invention is not particularly limited as long as the genome editing enzyme is an enzyme capable of forming a complex together with the guide RNA and editing a genome in a site-specific manner, but is represented by a nuclease (typically an endonuclease). The endonuclease includes, but is not limited to, for example, Cas9 proteins and Cpf1 proteins. Alternatively, it can also be considered to utilize, for example, a fusion protein of a Cas9 protein the nuclease activity of which is partly or completely eliminated and a deaminase. Hence, the “editing” of genomes in the present invention includes not only cleaving but also other modifications of genomes such as deamination as well as alteration (for example, mutagenesis) of genomes via these modifications

As the Cas9 proteins, those derived from various sources are known (for example, U.S. Pat. Nos. 8,697,359, 8,865,406, International Publication No. 2013/176772, and the like), and any of these may be utilized. From the viewpoint of the limitation in length of genes that can be expressed from virus vectors, a Cas9 protein having a relatively small molecular weight is preferable. Such a Cas9 protein includes a Cas9 protein derived from Staphylococcus aureus (SaCas9). The amino acid sequences and the base sequences of the Cas9 proteins are registered in a public database, for example, GenBank (http://www.ncbi.nlm.nih.gov) (for example, accession number: J7RUA5, WP_010922251, and the like, SEQ ID NO: 1, 5).

It is preferable that in the present invention, as the Cas9 protein, a protein comprising an amino acid sequence of SEQ ID NO: 2 OR 6 or consisting of the amino acid sequence be utilized. In addition, as the Cas9 protein in the present invention, a mutant comprising an amino acid sequence obtained by deleting, substituting, adding, or inserting one or a plurality of amino acids in a natural amino acid sequence may be used. Here, the “plurality of” is 1 to 50, preferably 1 to 30, and further preferably 1 to 10. Moreover, the Cas9 protein in the present invention also includes a polypeptide comprising an amino acid sequence having a sequence identity of 80% or more, more preferably 90% or more, further preferably 95% or more, and most preferably 99% or more with the amino acid sequence of SEQ ID NO: 2 OR 6 or consisting of the amino acid sequence as long as the original protein activity is maintained. The comparison between amino acid sequences can be conducted by a publicly-known approach, and can be conducted using, for example, BLAST (Basic Local Alignment Search Tool) at the National Center for Biological Information of the United States or the like with the default settings, for example.

As the Cpf1 protein, various types are known. Those described in documents (Zetsche, B. et al. Cell 163 (3), 759-71 (2015), Endo et al. Sci. Rep. 6, 38169 (2016)) can be utilized. Preferably used is a Cpf1 protein (LbCpf1, AsCpf1, FnCpf1) derived from Lachnospiraceae bacterium, Acidaminococcus sp., or Francisella novicida. The amino acid sequences of these Cpf1 proteins are registered in a public database, for example, GenBank (http://www.ncbi.nlm.nih.gov) (for example, accession number: WP_021736722, WP_035635841, and the like). In addition, as in the case of the Cas9 protein, it is possible to use a mutant comprising an amino acid sequence obtained by deleting, substituting, adding, or inserting one or a plurality of amino acids in a natural amino acid sequence. As a result of cutting the target double-stranded DNA, the Cas9 proteins generate blunt ends while the Cpf1 proteins generate staggered ends.

The “split genome editing enzymes” in the present invention are not particularly limited as long as the split genome editing enzymes can be expressed by the virus vectors and can reproduce the function as a genome editing enzyme when assembled in a cell. A genome editing enzyme is usually split into two fragments, but may be split into three fragments. An SaCas9 protein can be split into two fragments, an N-terminal side (amino acid residue 739) and a C-terminal side (amino acid residue 314) by a publicly-known method, for example, a method described in a document (Nishimasu et al. Cell, 162: 1113-1126 (2015)). In addition, for a Cas9 protein derived from Streptococcus pyogenes (SpCas9), for example, there are reported methods, that is, a method for splitting the SpCas9 into two fragments, a N-terminal side (amino acid residue 714) and a C-terminal side (amino acid residue 654) (Zetsche et al. Nat Biotechnol, 33: 139-142 (2015)), and a method for splitting the SpCas9 into a nuclease lobe (positions 1 to 57+GSS+positions 729 to 1368) containing the N-terminal and the C-terminal and a DNA recognition lobe (positions 56 to 714) therebetween (Wright et al. Proc Natl Acad Sci USA, 112, 2984-2989 (2015)). To the split genome editing enzyme, a nuclear localization signal and a tag, for example, may be attached.

The “guide RNA” in the present invention comprises: a base sequence complementally with a base sequence in the target DNA region; and a base sequence interacting with the above-described genome editing enzyme. The “target DNA region” in the present invention means a region containing a site where a target gene alteration occurs on a genome DNA of a living organism and is a region consisting of normally 17 to 30 bases, and preferably 17 to 20 bases.

When the genome editing enzyme is a Cas9 protein or a Cpf1 protein, the above region is preferably selected from regions adjacent to a PAM (proto-spacer adjacent motif) sequence. Typically, the cleavage of the target DNA in a site-specific manner occurs at a position determined by both of the complementarity of the base-pair formation between the guide RNA and the target DNA and the adjacent PAM. Although this depends on the type and origin of a nuclease, PAM typically is “5′-NNGRRT (N is any base)-3′” or “5′-NNGRR (N is any base)-3′” in the case of the SaCas9 protein, is “5′-NGG (N is any base)-3′” in the case of the SpCas9 protein, and is “5′-TTN (N is any base)-3′” or “5′-TTTN (N is any base)-3′” in the case of the Cpf1 protein. Note that it is also possible to alter the PAM recognition by alteration of a protein (for example, introduction of mutation) (Benjamin, P. et al., Nature 523, 481-485 (2015), Hirano, S. et al., Molecular Cell 61, 886-894 (2016)). This makes it possible to increase the options for the target DNA.

The guide RNA forms a complex together with a genome editing enzyme because the guide RNA comprises the base sequence (protein-binding segment) interacting with the genome editing enzyme (that is, bound to the genome editing enzyme by non-covalent bonding interaction). In addition, the guide RNA provides target specificity to the complex because the guide RNA comprises the base sequence (DNA-targeting segment) complementary with the base sequence of the target DNA region. As described above, the genome editing enzyme itself binds to the protein-binding segment of the guide RNA to be introduced to the target DNA region and edits (for example, in a case where the genome editing enzyme is a nuclease, cleaves) the target DNA owing to its activity.

In the case of the CRISPR/Cas9 system, the guide RNA is a combination of a crRNA fragment and a tracrRNA fragment. The crRNA fragment at least comprises a base sequence complementary with the base sequence of the target DNA region and a base sequence capable of interacting with the tracrRNA fragment in this order from the 5′ side. The tracrRNA fragment comprises, on the 5′ side, a base sequence capable of binding to (hybridizing with) a base sequence of part of the crRNA fragment. The crRNA fragment forms a double-stranded RNA together with the tracrRNA fragment at the base sequence capable of interacting with the tracrRNA fragment. The double-stranded RNA thus formed interacts with the Cas9 protein. In this way, the Cas9 protein is guided to the target DNA region. The crRNA fragment and the tracrRNA fragment can be fused to be expressed as a single molecule. On the other hand, in the case of the CRISPR/Cpf1 system, the guide RNA means the crRNA fragment and the tracrRNA fragment is unnecessary. The Cpf1 protein is guided to the target DNA region by interaction of the crRNA fragment with the Cpf1 protein.

To target a plurality of DNA regions and to target a plurality of portions in the same DNA region, a plurality of types of guide RNAs may be used. When an nCas9 protein is utilized, for example, it is possible to use a plurality of types of guide RNAs targeting one portion (two portions in total) of each of the strands in the double-strands of the target DNA region.

A plant positive-sense single-stranded RNA virus vector basically comprises: a replicase necessary for proliferation of the virus; a movement protein necessary for the virus to move from one cell to another in an infected plant; and a polynucleotide encoding an envelope protein, which protects the virus gene from attack from the surroundings. In the plant positive-sense single-stranded RNA virus vector used in the present invention, the polynucleotide encoding a split genome editing enzyme can be inserted into various positions as long as the replication and intercellular movement of the virus genome are not inhibited, and for example, can be inserted downstream of a polynucleotide encoding a movement protein. The polynucleotide may be inserted by substitution with a polynucleotide encoding a protein (for example, envelope protein) of the virus itself.

In the combination of plant positive-sense single-stranded RNA virus vectors used in the present invention, the guide RNA is arranged in at least one of the virus vectors. The guide RNA may be arranged in a plurality of virus vectors or may be arranged all the virus vectors.

The guide RNA may be arranged, for example, downstream of a polynucleotide encoding the split genome editing enzyme. A polynucleotide encoding a self-cleaving ribozyme is preferably bound to the 5′ terminal of a polynucleotide encoding the guide RNA, and in the transcription product, cleavage occurs on the 5′ side of the guide RNA due to the action of the ribozyme. The self-cleaving ribozyme is preferably a hammerhead ribozyme (Hamman et al. RNA 18: 871-885 (2011)). In the hammerhead ribozyme, a polynucleotide encoding an RNA complementary with the 5′-terminal region of the guide RNA is bound to the 5′ terminal of the polynucleotide encoding the ribozyme. Employing this structure allows the RNA added to the 5′ side of the self-cleaving ribozyme and the 5′ terminal of the guide RNA to be hybridized in the transcription product and cleavage occurs on the 5′ side of the guide RNA due to the action of the ribozyme. In addition, a polynucleotide encoding a self-cleaving ribozyme is preferably bound to the 3′ terminal of the polynucleotide encoding the guide RNA, and in the transcription product, cleavage occurs on the 3′ side of the guide RNA due to the action of the ribozyme. The self-cleaving ribozyme is preferably a hammerhead ribozyme or a hepatitis delta virus ribozyme (Webb and Luptak RNA biology 8: 5, 719-727). In the hammerhead ribozyme, a polynucleotide encoding an RNA complementary with the 3′-terminal region of the guide RNA is bound to the 3′ terminal of the polynucleotide encoding the ribozyme. Employing this structure allows the RNA added to the 3′ side of the self-cleaving ribozyme and the 3′ terminal of the guide RNA to be hybridized in the transcription product and cleavage occurs on the 3′ side of the guide RNA due to the action of the ribozyme. When a hepatitis delta virus ribozyme is employed, the RNA complementary with the 3′-terminal region of the guide RNA is unnecessary, and cleavage occurs at the 5′ terminal of the ribozyme. As a result of the actions of these ribozymes, unnecessary sequences are removed from the 5′ side and/or 3′ side of the guide RNA, allowing the guide RNA to efficiently function.

When a hammerhead ribozyme is used in the present invention, the “RNA comlementary with the 5′-terminal region of the guide RNA” or the “RNA comlementary with the 3′-terminal region of the guide RNA” to be arranged in a portion to be cleaved each has length and sequence sufficient for the self-cleaving ribozyme to be cleaved at the 5′ side and the 3′ side of the guide RNA in the transcription product. However, since the transcription product in which cleavage has occurred cannot be replicated to be a genome RNA of the virus, it is not preferable that all the transcription products be cleaved, and it is preferable to leave some transcription products uncleaned. In other words, it is preferable that the lengths and sequences of the “RNA comlementary with the 5′-terminal region of the guide RNA” and the “RNA comlementary with the 3′-terminal region of the guide RNA” be selected so as to generate both of a transcription product which has been cleaved by the self-cleaving ribozyme and a transcription product which has not been cleaved. The ratio of transcription products cleaved on the 5′ side and/or 3′ side of the guide RNA to the total transcription products is preferably 1 to 70%, and more preferably 5 to 30%. The length is normally 3 to 10 bases but is not limited thereto. It is also possible for a person skilled in the art to adjust the ratio between cleaved transcription products and uncleaned transcription products by introducing a non-complementary base as necessary. Also when a hepatitis delta virus ribozyme is used, it is preferable to employ a hepatitis delta virus ribozyme having an appropriate sequence so as to make the ratio of cleaved transcription products to the total transcription products equal to the above-described ratio.

When the plant positive-sense single-stranded RNA virus vector is in the form of an RNA, it is possible to use, for example, an RNA product obtained by preparing an expression construct obtained by inserting the above-described exogenous gene into a cDNA corresponding to the viral genome RNA, followed by in vitro transcription. When the plant positive-sense single-stranded RNA virus vector is in the form of a DNA, it is possible to use, for example, an expression construct obtained by inserting the exogenous gene into a cDNA corresponding to the viral genome RNA.

When the plant positive-sense single-stranded RNA virus vector is used as a DNA vector (expression construct), the virus gene and the exogenous gene are usually bound to the downstream of an appropriate promoter that can be expressed in a plant. As the promoter, a publicly-known promoter such as a CaMV 35S promoter, a rice actin promoter, and an ubiquitin promoter can be used, for example. A terminator is usually bound to the downstream of these genes. The protein encoded by the exogenous gene may also be expressed as a fusion protein with a protein encoded by the virus gene via a recognition sequence of a sequence-specific protease, for example. In this case the fusion protein is cleaved to generate the protein encoded by the exogenous gene owing to the action of the protease.

In the present invention, the plant positive-sense single-stranded RNA virus vectors prepared as described above are introduced into a plant cell. The plant cell may be selected from host plant cells that the virus vector can infect, and includes cells of various plants such as vegetables, fruits, and horticultural crops. The “plant” includes, but is not limited to, the Solanaceae (for example, tobacco, eggplant, potato, bell pepper, tomato, pepper, and petunia), the Poaceae (rice, barley, rye, Japanese millet, sorghum, and corn), the Brassicaceae (for example, daikon radish, turnip rape, cabbage, Arabidopsis thaliana, Japanese horse-radish, and shepherd's-purse), the Rosaceae (for example, Japanese apricot, peach, apple, pear, strawberries, and rose), the Fabaceae (for example, soybean, adzuki bean, common bean, green pea, broad bean, peanut, clover, and burr medic), the Cucurbitaceae (for example, sponge gourd, squash, cucumber, watermelon, melon, and zucchini), the Lamiaceae (for example, lavender, Japanese mint, and Japanese basil), the Liliaceae (for example, green onion, garlic, lily, and tulip), the Chenopodiaceae (for example, spinach), the Apiaceae (for example, wild celery, carrot, mitsuba, and celery), the Asteraceae (for example, chrysanthemum, lettuce, and artichoke), the Orchidaceae (for example, moth orchids and cattleya orchids), the Convolvulaceae (for example, sweet potato), the Araceae (for example, Colocasia esculenta, taro, and konjac), and the like. In addition, the “plant cell” includes cells inside the plant besides culture cells. Further, the “plant cell” includes various forms of plant cells, for example, suspended culture cells, protoplasts, leaf sections, calli, immature embryos, pollens, and the like.

As the method for introducing the plant positive-sense single-stranded RNA virus vectors into a plant cell, a publicly-known method such as a rubbing inoculation method and a particle gun method, for example, may be utilized.

In the plant cell in which the combination of plant positive-sense single-stranded RNA virus vectors has been introduced, the split genome editing enzymes are expressed and assembled to form a functional genome editing enzyme. The functional genome editing enzyme and the guide RNA form a complex, and the genome is edited (for example, cleaved when the genome editing enzyme is a nuclease) in a site-specific manner by the complex.

In the present invention, it is possible to produce a plant in which a genome is edited in a site-specific manner by regenerating the plant from a plant cell in which the combination of plant positive-sense single-stranded RNA virus vectors has been introduced. As the method for obtaining individuals by redifferentiating the tissue of the plant in tissue cultures, a method established in the art can be utilized (Tabei Y. Ed., “Protocols of Plant Transformation, Kagaku-Dojin Publishing Company, INC pp. 340-347, 2012). Once a plant is obtained as described above, it is also possible to obtain a progeny from the plant by sexual reproduction or asexual reproduction. In addition, it is also possible to obtain propagation materials (for example, seeds, fruits, spikes, stubs, calli, protoplasts, and the like) from the plant or a progeny or a clone thereof, and to produce the plant in mass from these propagation materials. The present invention encompasses plants obtained by the method of the present invention, progenies and clones of the plants, as well as propagation materials of the plants, the progenies and clones of the plants.

The present invention also provides a kit for use in the above-described method of the present invention.

The kit comprises a combination of a plurality of types of plant positive-sense single-stranded RNA virus vectors having the following characteristics (a) and (b).

(a) each of the virus vectors contains a polynucleotide encoding a split genome editing enzyme

(b) at least one of the virus vectors contains a polynucleotide encoding a guide RNA or a portion for inserting the polynucleotide

The kit may further comprise one or a plurality of additional elements. The additional elements include, but are not limited to, for example, reagents for introducing vectors into cells, diluted buffer solutions, wash buffers, growth media, control reagents (for example, control vectors), and the like. The kit comprises an instruction manual in general.

### EXAMPLES

Hereinafter, the present invention will be described in more detail based on Examples; however, the present invention is not limited to Examples described below.

Note that materials used in Examples are as described below.
In Vitro Synthesis of Virus RNA: pTLW3 (Kubota et al. J Virol. 77: 11016-11026 (2013), ToMV, plasmid DNA), pP2C2S (Chanpman et al., Plant J. 2: 549-557 (1992), PVX, plasmid DNA), Mlu1 (Takara Bio Inc.), Spe1 (NEB), BstNI (NEB), AmpliCap-Max™ T7 High Yield Message Maker Kit (CELLSCRIPT), DNAiso (Takara Bio Inc.)
Plants Under Test: Nicotiana benthamiana, Nicotiana tabacum
Others: Carborundum (Nacalai Tesque), KOD plus neo (TOYOBO), DNA Ligation Kit <Mighty Mix> (Takara Bio Inc.)

### Example 1

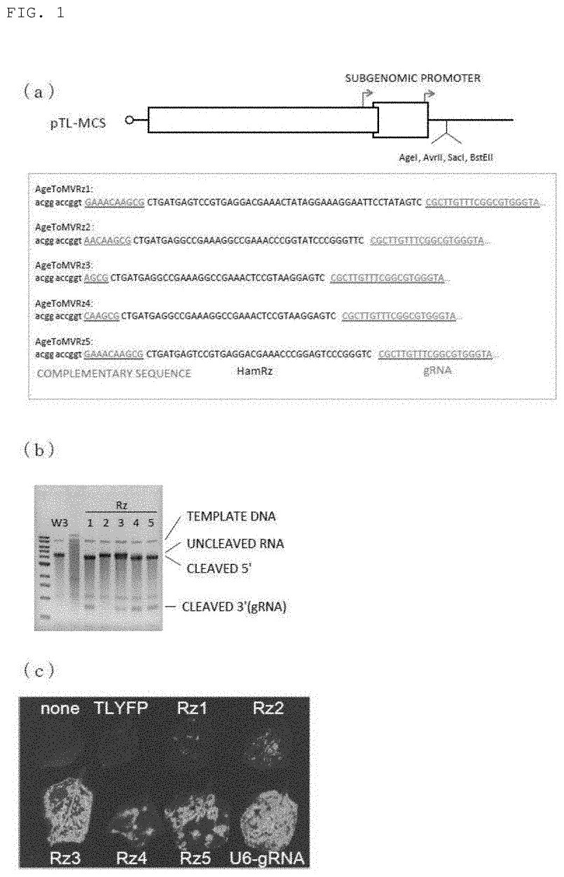

ToMV vectors in each of which a gRNA was bound to the 3′ side of a self-cleaving ribozyme was constructed such that the 5′ side of a guide RNA (gRNA) was cleaved by the action of the self-cleaving ribozyme (FIG. 1a). As the self-cleaving ribozyme, HamRz (CTGATGAGGCCGAAAGGCCGAAACTCCGTAAGGAGTC/SEQ ID NO: 3) was used and sequences of various lengths that were complementary with the 5′ side of the gRNA were arranged on the 5′ side. To the ToMV vectors thus constructed, in vitro transcription (at 37° C. for 2.5 hours) was conducted, and virus RNAs which were transcription products thus obtained were developed through agarose electrophoresis. As a result, it was revealed that the cleavage efficiency of the 5′ side of the gRNA varied depending on the difference of the sequence arranged on the 5′ side of the ribozyme (FIG. 1b).

The above-described ToMV vectors were inoculated into leaves of Nicotiana benthamiana in which pDe-CAS9, which expresses SpCas9 (Fauser et al. Plant J. 79 (2): 348-359 (2014)), and a target sequence (SEQ ID NO: 4) were transiently introduced, by the agroinfiltration method. If the target sequence is cleaved and repaired by a complex of SpCas9 and gRNA, the LUC gene is expressed. When the LUC activity after 6 days was detected, a strong LUC activity was detected in a case where Rz3 was used (FIG. 1c). Note that “TLYFP” represents a negative control that did not have a gRNA and “U6-gRNA” represents a positive control that expressed a gRNA from an U6 promoter through agroinfiltration.

### Example 2

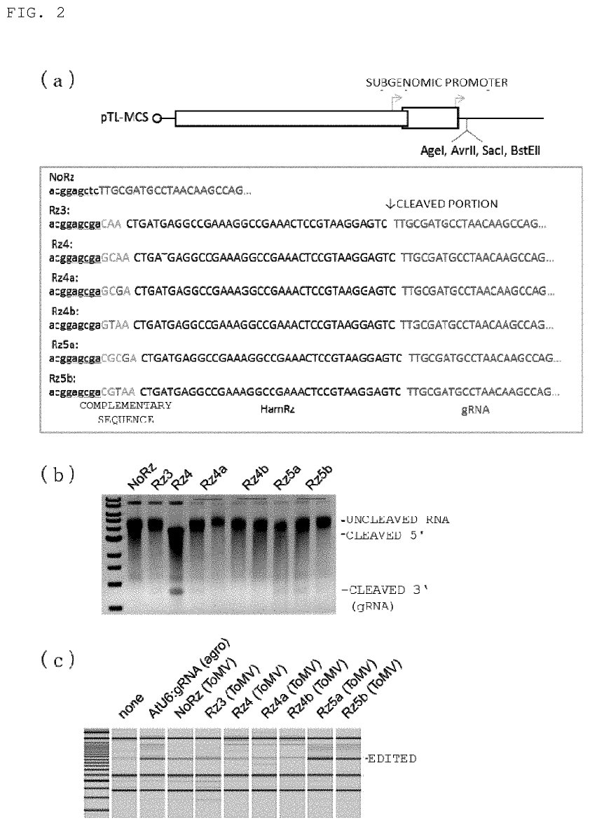

A gRNA targeting a PDS gene of tobacco was inserted into the ToMV vectors. On the 5′ side of the self-cleaving ribozyme HamRz, sequences of various lengths that were complementary with the 5′ side of the gRNA were arranged (FIG. 2a; In some sequences, bases that were not complementary with the 3′ side of the gRNA were also introduced). To the ToMV vectors thus constructed, in vitro transcription (at 37° C. for 2.5 hours) was conducted, and virus RNAs which were transcription products thus obtained were developed through agarose electrophoresis. As a result, it was revealed that the cleavage efficiency of the 5′ side of the gRNA varied depending on the difference of the sequence arranged on the 5′ side of the ribozyme (FIG. 2b).

The above-described ToMV vectors were inoculated into leaves of Nicotiana benthamiana in which a plasmid based on pRI201-AN, which expresses SaCas9 (Kaya et al. Sci Rep 6: 26871 (2016)), were transiently introduced, by the agroinfiltration method. The genome DNAs were extracted after 7 days, and whether the editing occurred or not was examined by the CAPS method. It was found that genome editing occurred efficiently in Rz5a and Rz5b having appropriate cleavage efficiencies (FIG. 2c). Note that “NoRz” represents a negative control that did not have HamRz and “AtU6:gRNA” represents a positive control that expressed a gRNA from an U6 promoter through agroinfiltration.

### Example 3

From Examples 1 and 2 described above, it was revealed that the genome editing efficiency was significantly improved by adjusting the length and type of a sequence in sequences complementary with the gRNA arranged on the 5′ side of HamRz to appropriately maintain the base pair formation efficiency with the gRNA (FIGS. 1 and 2). In view of this, subsequently, the construction of vectors in which split Cas9 genes were inserted was conducted.

(1) Cloning of Cas9

The SaCas9 gene (3159 bp) isolated from Staphylococcus aureus was split into the N-terminal side (2217 bp, referred to as “N739”) and the C-terminal side (942 bp, referred to as “C740”). These were cloned using KOD plus neo, and the resultants were introduced to the downstream of each of subgenomic promoters pTLW3 and pP2C2S to create pTL-739N, pTL-740C, pPVX-739N, and pPVX-740C. Moreover, a gRNA sequence targeting the above-described tobacco PDS gene was bound to the downstream of the split Cas9 in each of pTL-739N and pTL-740C via HamRz, which is a self-cleaving ribozyme (pTL-739N-Rz5a and pTL-740C-Rz5a).

(2) Virus Infection

Virus RNAs were synthesized using AmpliCap-Max™ T7 High Yield Message Maker Kit with plasmid DNAs, which were opened by restriction enzymes (MluI for ToMV and SpeI for PVX), as templates. The RNAs thus synthesized were mixed in two combinations of [TL-739N-Rz5a and PVX-740C] and [TL-740C-Rz5a, PVX-739N], followed by rubbing together with Carborundum to infect tobacco (Nicotiana benthamiana or Nicotiana tabacum) leaves (about 4 weeks old, fifth leaves).

(3) Extraction of Genome of Tobacco, CAPS Analysis

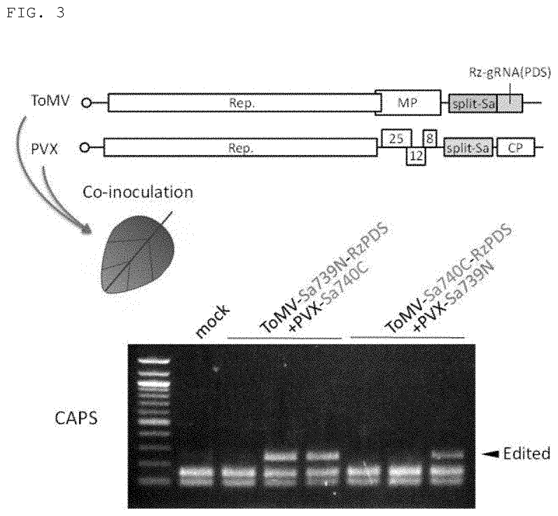

Leaves of Nicotiana benthamiana 12 days after the inoculation were recovered as samples, which were frozen and crushed with liquid nitrogen, and then genomes were extracted using 500 μL of DNAiso. About 250 bp fragment containing a gRNA target portion was amplified through PCR using the genomes as templates. Subsequently, 2 μL of the PCR reaction solution was digested with a restriction enzyme BstNI, followed by CAPS analysis. As a result of the CAPS analysis, the genome editing of the target portion was observed in any combination of the viruses (FIG. 3).

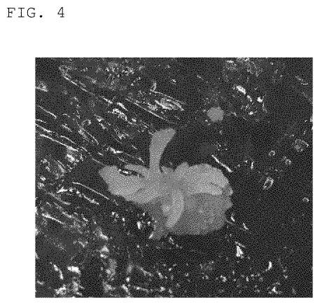

In addition, the infected leaves of Nicotiana tabacum were differentiated (callus formation) in accordance with the reported method (Ohshima et al. Plant Cell 2: 95-106 (1990)), and then shoot regeneration was attempted. As a result of the attempt, white shoot indicating that the PDS gene was knocked out was observed (FIG. 4).

### Example 4

When the genome editing was conducted by expressing split SaCas9 from the virus vector in Example 3, the ribozyme inserted in the virus genome cleaves the 5′ side of the gRNA to supply the gRNA. However, even in this case, a sequence derived from the virus is attached to the 3′ side of the supplied gRNA. In view of this, in the present Example, a ribozyme that causes cleavage at a low efficiency on the 3′ side of a gRNA was further arranged to study whether the genome editing efficiency was changed.

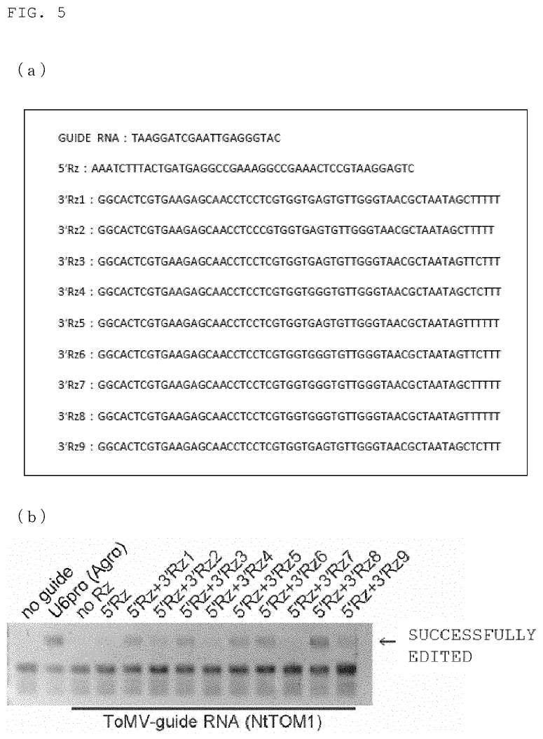

Specifically, a gRNA targeting a tobacco TOM1 gene was inserted into the ToMV vector. Ribozymes were arranged on either side of the gRNA. At this time, the ribozyme that cleaves the 5′ side of the gRNA was fixed (5′Rz) while ribozymes having various cleavage efficiencies were arranged on the 3′ side (FIG. 5a). The above-described ToMV vectors were inoculated into leaves of Nicotiana benthamiana in which a plasmid based on pRI201-AN, which expresses SaCas9 (Kaya et al. Sci Rep 6: 26871 (2016)), was transiently introduced, by the agroinfiltration method, and whether genome editing occurred or not was examined by the CAPS method as in the case of Example 2.

As a result, the genome editing efficiency was significantly improved by arranging an appropriate ribozyme on the 3′ side as well (FIG. 5b). The genome editing efficiency greatly varied depending on a ribozyme to be added, and Rz8 exhibited the highest genome editing efficiency.

### Example 5

In Example 3, split SaCas9 was used as a genome editing enzyme that was expressed from the virus vectors. In the present Example, examination was similarly conducted but using split SpCas9 in order to confirm the versatility of this technique.

Specifically, SpCas9 isolated from Streptococcus pyogenes was split into the N-terminal side (“positions 1 to 714”; 2280 bp; Sp_N714) and the C-terminal side (“positions 715 to 1368”; 1998 bp; Sp_C715) by referring to an example using animal cells (Zetsche et al. Nat Biotechnol, 33: 139-142 (2015)). In addition, a method for splitting SpCas9 into a nucleic acid recognition domain (“positions 56 to 714”; 1977 bp; Sp_α-Helical) and a nucleic acid degradation domain (“positions 1 to 57+GSS+positions 730 to 1368”; 2100 bp; Sp_Nuclease) (“partially modified version of Wright et al. Proc Natl Acad Sci USA, 112, 2984-2989 (2015)”) was also examined. A nuclear localization signal code sequence was added to each of the 5′ sides of Sp_N714 and Sp_α-Helical and to each of the 3′ sides of Sp_C715 and Sp_Nuclease. All of the SpCas9s were amplified through PCR using KOD plus neo. Sp_N714 and Sp_α-Helical were each substituted with an envelope protein gene of pTLW3 (ToMV) while Sp_C715 and Sp_Nuclease were each introduced into SalI and EcoRV cleaved portions of pP2C2S (PVX). Furthermore, a guide RNA sequence targeting a tobacco RTS3 gene was coupled to the downstream of each of Sp_C715 and Sp_Nuclease via HamRz (CTGATGAGGCCGAAAGGCCGAAACTCCGTAAGGAGTC/SEQ ID NO: 3), which is a self-cleaving ribozyme.

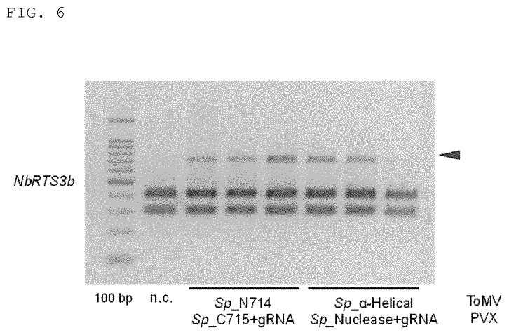

Both virus vectors were co-inoculated in Nicotiana benthamiana, and whether genome editing occurred or not was examined by the CAPS method. As a result, the genome editing of the target portion was able to be observed in any combination of viruses (FIG. 6). Therefore, it was confirmed that split SpCas9 can also be utilized in the present invention.

### INDUSTRIAL APPLICABILITY

As described above, the present invention makes it possible to conduct genome editing of a plant using plant virus vectors without integrating a genome editing enzyme gene into a plant genome. According to the present invention, since it is possible to efficiently produce agricultural crops having useful characters, for example, the present invention can be industrially utilized mainly in the field of agriculture.

### [Sequence Listing Free Text]

### SEQ ID NO: 3

artificially synthesized ribozyme sequence (HamRz)

### SEQ ID NO: 4

Luc gene in which a Cas9 target sequence is inserted

inserted sequence

Cas9 target sequence

### [Sequence Listing] IBPF17-539WO-seq-fin.txt

1. A method for producing a plant cell in which a genome is edited in a site-specific manner, comprising:
introducing a combination of a plurality of types of plant positive-sense single-stranded RNA virus vectors having characteristics (a) and (b) into a plant cell, where
(a) each of the virus vectors contains a polynucleotide encoding a split genome editing enzyme, and
(b) at least one of the virus vectors contains a polynucleotide encoding a guide RNA; and
allowing a complex containing an assembly of the split genome editing enzymes and the guide RNA to be formed in the plant cell and allowing the complex to edit the genome in a site-specific manner.

2. A method for producing a plant in which a genome is edited in a site-specific manner, comprising:
introducing a combination of a plurality of types of plant positive-sense single-stranded RNA virus vectors having characteristics (a) and (b) into a plant cell, where
(a) each of the virus vectors contains a polynucleotide encoding a split genome editing enzyme, and
(b) at least one of the virus vectors contains a polynucleotide encoding a guide RNA; and
allowing a complex containing an assembly of the split genome editing enzymes and a guide RNA to be formed in a plant cell, allowing the complex to edit the genome in a site-specific manner, and regenerating a plant from the plant cell.

3. The method according to claim 1, wherein
the combination of plant positive-sense single-stranded RNA virus vectors includes a combination of a Tobamovirus vector and a Potexvirus vector.

4. The method according to claim 1, wherein
the genome editing enzyme is a Cas9 protein or a Cpf1 protein.

5. The method according to claim 1, wherein
a polynucleotide encoding a self-cleaving ribozyme is bound to a 5′ terminal of the polynucleotide encoding a guide RNA.

6. The method according to claim 1, wherein
a polynucleotide encoding a self-cleaving ribozyme is bound to a 3′ terminal of the polynucleotide encoding a guide RNA.

7. A kit for use in the method according to claim 1, comprising:
a combination of a plurality of types of plant positive-sense single-stranded RNA virus vectors having characteristics (a) and (b), where
(a) each of the virus vectors contains a polynucleotide encoding a split genome editing enzyme, and
(b) at least one of the virus vectors contains a polynucleotide encoding a guide RNA or a portion for inserting the polynucleotide.

8. The kit according to claim 7, wherein
the combination of plant positive-sense single-stranded RNA virus vectors includes a combination of a Tobamovirus vector and a Potexvirus vector.

9. The kit according to claim 7, wherein
the genome editing enzyme is a Cas9 protein or a Cpf1 protein.

10. The kit according to claim 7, wherein
a polynucleotide encoding a self-cleaving ribozyme is bound to a 5′ terminal of the polynucleotide encoding a guide RNA.

11. The kit according to claim 7, wherein
a polynucleotide encoding a self-cleaving ribozyme is bound to a 3′ terminal of the polynucleotide encoding a guide RNA.
